(19) United States
(12) Patent Application Publication
(10) Pub. No.: US 2026/0013885 A1
(43) Pub. Date: Jan. 15, 2026

(54) MICRO PROBE FOR PLAQUE REMOVAL

(71) Applicant: Maurice A Khawam, Lakewood, CA (US)

(72) Inventor: Maurice A Khawam, Lakewood, CA (US)

(21) Appl. No.: 19/268,867

(22) Filed: Jul. 14, 2025

Related U.S. Application Data
(60) Provisional application No. 63/670,704, filed on Jul. 12, 2024.

(51) Int. Cl. A61B 17/22; A61B 17/00; A61B 17/32

(52) U.S. Cl. CPC A61B 17/22; A61B 17/00234; A61B 17/32; A61B 2017/00238; A61B 2017/00292; A61B 2017/00561; A61B 2017/22038; A61B 2017/22079; A61B 2017/22082; A61B 2017/320008; A61B 2217/005

(57) ABSTRACT

The present invention is a micro probe for plaque removal. In particular, the present invention is directed to a micro probe having a needle tip, an opening and perimeter openings for plaque removal. The micro probe is attached to a guide wire in a catheter and the catheter is attached to a pump. The needle tip is at the first end and the second end is attached to the guide wire and is insertable and retractable into the catheter. Each perimeter opening has a sharp edge and a blunt opposite edge and the micro probe can be rotated by the guide wire. The opening can also contain a dry ice pellet that can be impelled by the pump, or the pump can impel a solution through the catheter to the micro probe and aspirate the solution from the opening of the microprobe.

This patent application claims the benefit of a provisional patent application, U.S. Pat. Appl. No. 63/670,704, filed Jul. 12, 2024, which is included by reference in its entirety.

### TECHNICAL FIELD

The present invention is a micro probe for plaque removal. In particular, the present invention is directed to a micro probe having a needle tip, an opening and perimeter openings for plaque removal.

### BACKGROUND ART

Heart disease is one of the leading causes of death in the United States and is responsible for about 1 in 5 deaths. In 2024, approximately 931,578 people in the USA died from heart disease. The age-adjusted death rate from cardiovascular disease also increased in 2024 to 233.3 per 100,000, a 4.0% rise from the previous year.

Heart disease affects a significant portion of the population, with 48.6% of US adults having some form of cardiovascular disease between 2017 and 2020. Heart disease and stroke are also some of leading causes of death globally. According to the CDC, currently, one person dies every 33 seconds from cardiovascular disease.

In the United States, heart disease was associated with costs of approximately $252.2 billion from 2019 to 2020. This includes the cost of health care services, medicines, and lost productivity due to death. The direct and indirect costs of total cardiovascular disease (CVD) were estimated at $417.9 billion between 2020 and 2021. This number is expected to increase significantly in the years to come.

Heart disease is generally caused by the partial or complete occlusion of arteries that feed the heart with oxygenated blood and nutrients, elements that are essential for the heart to perform its primary function of pumping blood to the rest of the body. Blockages caused by plaque build-up in the liner of the endothelium of the heart is generally known as atherosclerosis. Plaque formation is often caused by poor diet, lack of exercise, smoking, family history and stress. Plaque generally results from inflammation and is often made up of fat, fibrin, calcium, cholesterol, and dead white blood cells. Plaque usually manifests in two varieties, a soft plaque made up of the substances listed above, or a hard plaque made up of calcium that adheres to the arterial walls.

Examples of prior art treatments for atherosclerosis are:
1. open heart surgery where an occluded artery is replaced with a vein from the patient's body e.g. a leg, or,
2. orbital or rotational atherectomy that shaves the calcium build-up in the occluded artery, or,
3. installation of a stent in the occluded artery that pushes the soft plaque outward and opens the lumen allowing blood flow to be reestablished to the heart ventricles.

Each of these prior art treatments has its own drawbacks. For example, open-heart surgery can be very expensive and dangerous. Moreover, plaque can start forming again after surgery. An atherectomy is not applicable in every case as some plaque does not provide enough traction to shave calcium build up. Stents can result in stenosis, namely the re-narrowing of the artery and this can lead to further complications.

Accordingly, a micro probe with plaque removal is needed that can remove soft and hard plaque from a patient's arterial walls.

### SUMMARY OF THE INVENTION

The present invention is a micro probe for plaque removal. In particular, the present invention is directed to a micro probe having a needle tip, an opening and perimeter openings for plaque removal. The micro probe is attached to a guide wire in a catheter and the catheter is attached to a pump.

The needle tip is at the first end and the second end is attached to the guide wire and is insertable and retractable into the catheter. Each perimeter opening has a sharp edge and a blunt opposite edge and the micro probe can be rotated by the guide wire. The opening can also contain a dry ice pellet that can be impelled by the pump or the pump can impel a solution through the catheter to the micro probe and aspirate the solution from the opening of the microprobe.

### BRIEF DESCRIPTION OF THE DRAWINGS

The objects and features of the present invention, which are believed to be novel, are set forth with particularity in the appended claims. The present invention, both as to its organization and manner of operation, together with further objects and advantages, may best be understood by reference to the following description, taken in connection with the accompanying drawings.

FIG. 1 is a cross-sectional view of an artery;

FIG. 2 is a cross-sectional view of an artery with an occlusion;

FIG. 4A is a side view of a preferred embodiment of the invention retracted into a catheter;

FIG. 4B is a side view of preferred embodiment of the invention extended out of a catheter;

FIG. 5A is a side perspective view of an alternative embodiment of the invention;

FIG. 5B is a side view of the embodiment in FIG. 5A;

FIG. 5C is another side view of the embodiment in FIG. 5A;

FIG. 5D is a top view of the embodiment in FIG. 5A;

FIG. 5E is a cross-sectional view along line E-E in FIG. 5C;

FIG. 7A is a side perspective view of an alternative embodiment of the invention;

FIG. 7B is a side view of the embodiment in FIG. 7A;

FIG. 7C is another side view of the embodiment in FIG. 7A;

FIG. 7D is a top view of the embodiment in FIG. 7A;

FIG. 7E is a cross-sectional view along line C-C in FIG. 7C;

FIG. 8A is a side perspective view of an alternative embodiment of the invention;

FIG. 8B is a side view of the embodiment in FIG. 8A;

FIG. 8C is another side view of the embodiment in FIG. 8A;

FIG. 8D is a top view of the embodiment in FIG. 8A;

FIG. 8E is a cross-sectional view along line C-C in FIG. 8C;

FIG. 9A is a side perspective view of an alternative embodiment of the invention;

FIG. 9B is a side view of the embodiment in FIG. 9A;

FIG. 9C is another side view of the embodiment in FIG. 9A;

FIG. 9D is a partial view of the embodiment in FIG. 9C;

FIG. 9E is a top view of the embodiment in FIG. 9A;

FIG. 9F is a cross-sectional view along line C-C in FIG. 9C;

FIG. 10A is a side perspective view of an alternative embodiment of the invention;

FIG. 10B is a side view of the embodiment in FIG. 10A;

FIG. 10C is another side view of the embodiment in FIG. 10A;

FIG. 10D is a partial view of the embodiment in FIG. 10C;

FIG. 10E is a top view of the embodiment in FIG. 10A;

FIG. 10F is a cross-sectional view along line C-C in FIG. 10C;

FIG. 11A is a side perspective view of an alternative embodiment of the invention;

FIG. 11B is a side view of the embodiment in FIG. 11A;

FIG. 11C is another side view of the embodiment in FIG. 11A;

FIG. 11D is a partial view of the embodiment in FIG. 11C;

FIG. 11E is a top view of the embodiment in FIG. 11A;

FIG. 11F is a cross-sectional view along line C-C in FIG. 11C;

FIG. 12A is a side perspective view of an alternative embodiment of the invention;

FIG. 12B is a side view of the embodiment in FIG. 12A;

FIG. 12C is another side view of the embodiment in FIG. 12A;

FIG. 12D is a partial view of the embodiment in FIG. 12C;

FIG. 12E is a top view of the embodiment in FIG. 12A;

FIG. 12F is a cross-sectional view along line C-C in FIG. 12C;

FIG. 13 is a side view of another alternative embodiment of the invention;

FIG. 14 is a side cross-sectional view of another alternative embodiment of the invention;

FIG. 15 is a schematic of an alternative embodiment of the invention;

FIG. 16A is a partial side cross-sectional view of the embodiment of the invention in FIG. 15 during aspiration;

FIG. 16B is a partial side cross-sectional view of the embodiment of the invention in FIG. 15 during suction;

FIG. 17A is a partial cross-sectional view of the embodiment of the invention in FIG. 14 with scraping arms in the retracted position; and,

FIG. 17B is a partial cross-sectional view of the embodiment of the invention in FIG. 14 with scraping arms in the extended position.

### DETAILED DESCRIPTION OF THE PREFERRED EMBODIMENTS

The following description is provided to enable any person skilled in the art to make and use the invention and sets forth the best modes contemplated by the inventor of carrying out his invention. Various modifications, however, will remain readily apparent to those skilled in the art, since the general principles of the present invention have been defined herein specifically to provide a micro probe for plaque removal.

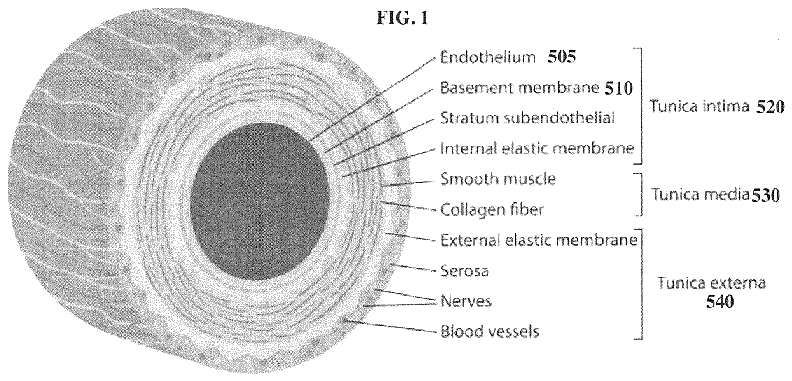

Referring now to FIG. 1, a cross section of a typical artery 500 with three main tunica 520, 530 and 540 is shown. An artery 500 has, among other things, an endothelium 505 and a basement membrane 510. Arteries are commonly 3-4 mm in diameter. When plaque forms, it typically forms between the endothelium 505 and the basement membrane 510.

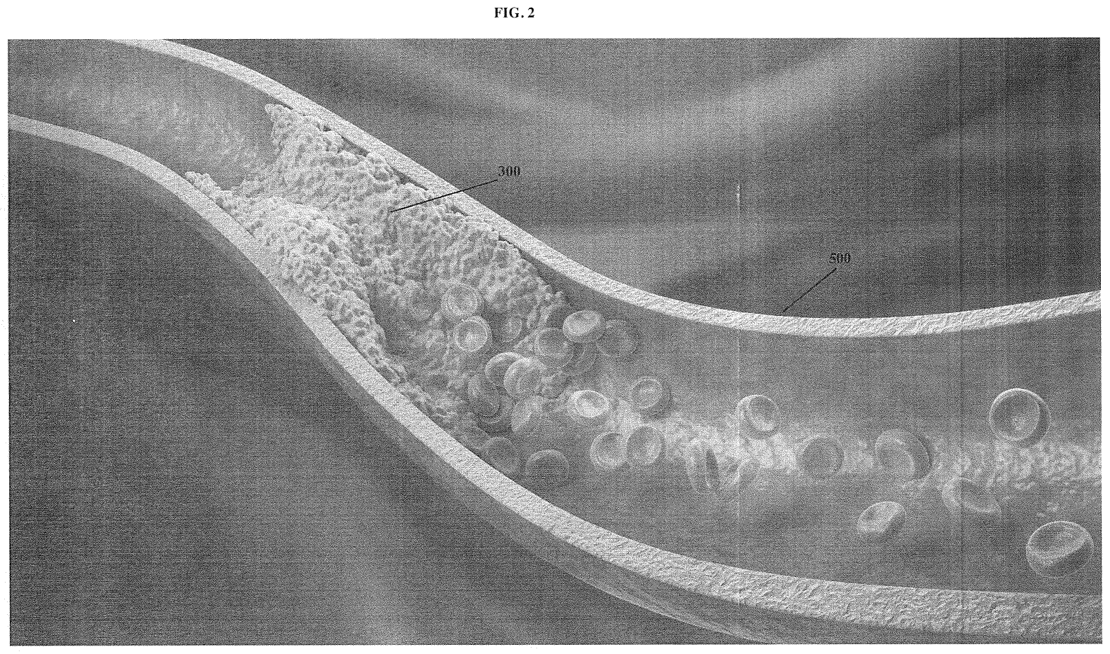

Referring to FIG. 2, an example of an occlusion 300 (or plaque accumulation) in an artery 500 is shown. Common catheter insertion points for a patient are at the groin, wrist and arm. A catheter 200 is typically inserted into a patient and guided by a guide wire 210 to a patient's heart where an artery has an occlusion. The guide wire 210 preferably comprises a fiber optic camera (not shown).

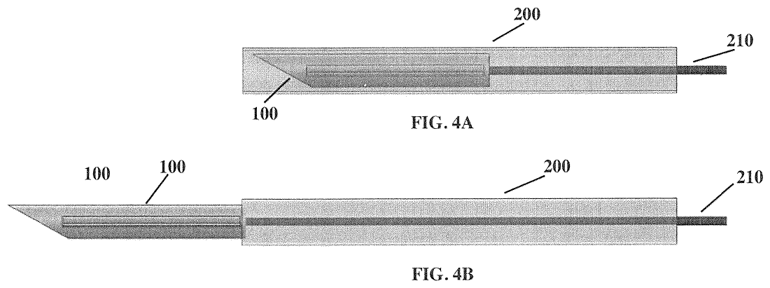

Referring now to FIGS. 4A and 4B, a preferred embodiment of a micro probe 100 is shown. FIG. 4A shows the micro probe 100 in a retracted position inside the catheter 200. The probe 100 is preferably guided through a patient in the retracted position to avoid causing unwanted cuts or damage to the patient. FIG. 4B shows the micro probe 100 in an extended position outside the catheter 200. The micro probe 100 is placed in the extended position and locked in place by a wheel (not shown) on the end of the guide wire 210. The micro probe 100 preferably has a needle tip 110. Dimensions of the probe 100 and catheter 200 can vary depending on the location and shape of an occlusion to be treated. A user, typically a surgeon, will then assess an optimal location in an endothelium and place the probe in the location to reach an occlusion, namely plaque trapped in an artery liner. However, a catheter with a diameter of 2.75 mm and 0.125 mm wall thickness can be used with a micro probe 100 with a diameter of 2.0 mm and 15 mm length. This proximity to the main blood stream allows the microprobe 100 to reach the occlusion 300 and suction it out as described herein.

The surgeon is generally looking for the portion of the endothelium 505 that is healthy and shows no signs of over-stretching. The surgeon then preferably applies suction to the catheter 200 from a suitable pump (not shown) capable of generating vacuum of 1 atm (14.7 psi or 760 mmHg). The pump preferably has settings that allow different suction levels to be applied as desired.

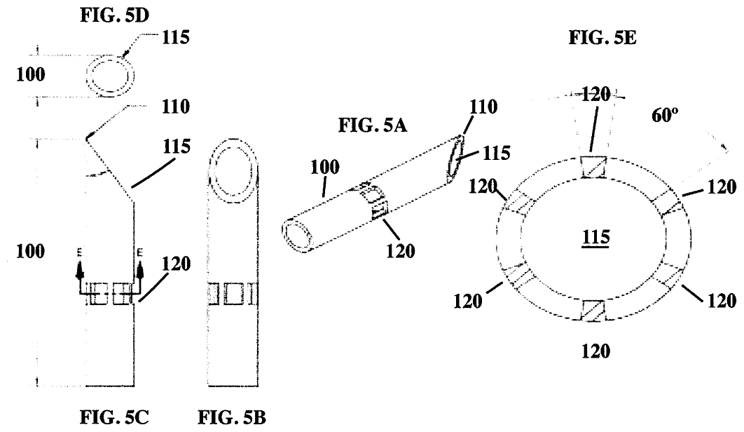

Another preferred embodiment of the probe 100 is shown in FIGS. 5A through 5E. The probe 100 preferably has an opening 115 on a first end of the probe 100 and a plurality of perimeter openings 120 that allow further suction and supplement the suction at the opening 115. The perimeter openings 120 preferably have sunken edges to slice suctioned plaque and expedite its removal. The embodiment shown in FIGS. 5A through 5E has six (6) openings 120 cut-out on a perimeter of the probe 100 to further aid in the aspiration of the plaque. These orifices 120 preferably have sharp edges to assist in the breakdown of the plaque. The orifices 120 are preferably cut-out at sixty (60) degree intervals on the perimeter of the probe 100 as shown in FIG. 5E. On a probe 100 with a 2 mm diameter, each orifice 120 is preferably 1.0 mm×1.0 mm. The opening 115 is most likely to draw the most plaque material during aspiration.

Referring now to FIGS. 6A through 6E, another alternative embodiment is shown. In FIGS. 6A through 6E, a shorter probe 100 such as with a length of 6 mm is preferably also useful for tighter lesion locations in a patient. The probe 100 has a needle tip 110 and opening 115. However, it has fewer openings 120 on the perimeter, e.g. three (3) as shown.

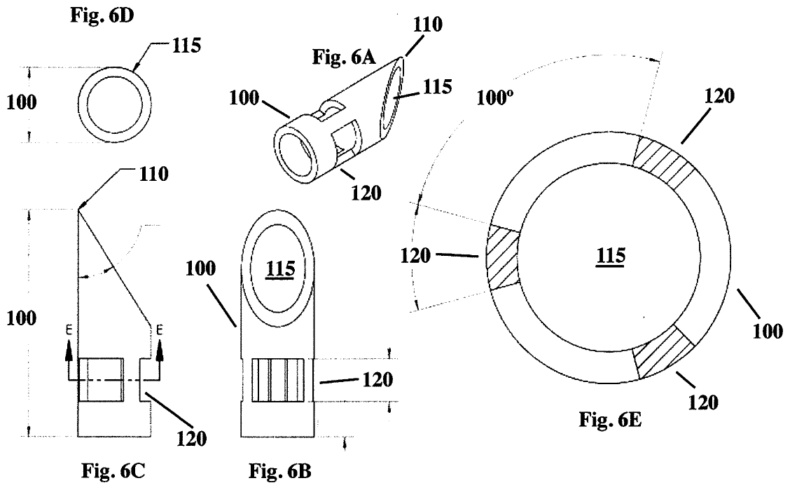

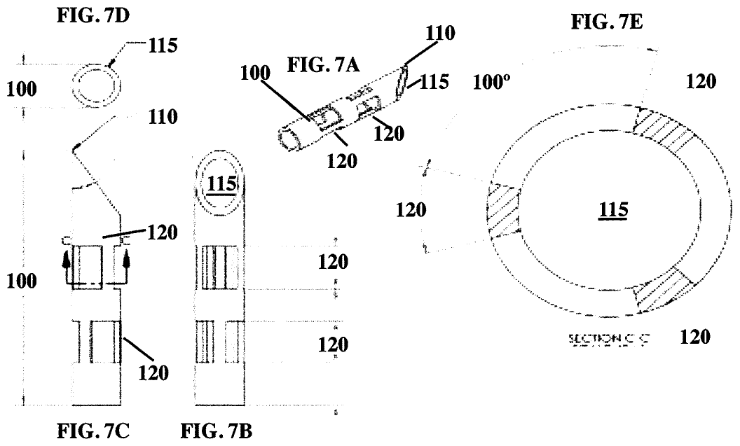

Referring now to FIGS. 7A through 7E, another alternative embodiment is shown. Like in FIGS. 6A through 6E, three (3) openings 120 are shown on the perimeter of the probe 100. However, in FIGS. 7A through 7E, the probe 100 is preferably longer, e.g. 12 mm, so two (2) sets of openings 120 are shown with staggered spacing around the perimeter. This configuration is possible in a shorter probe 100, e.g. 6 mm in length, as shown in FIGS. 8A through 8E.

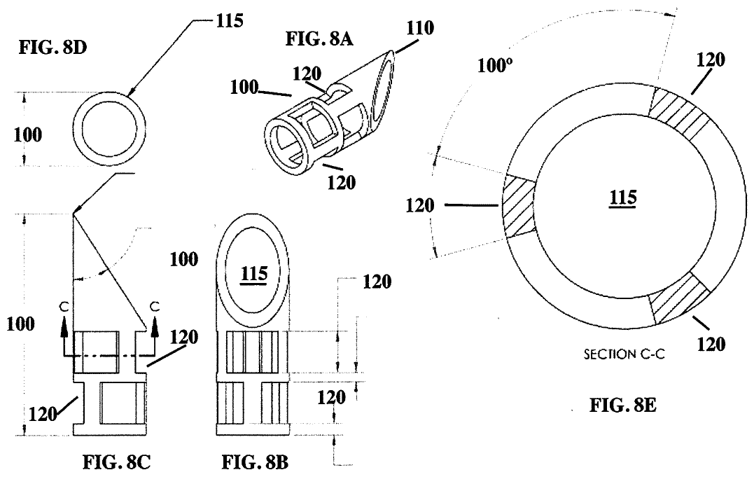

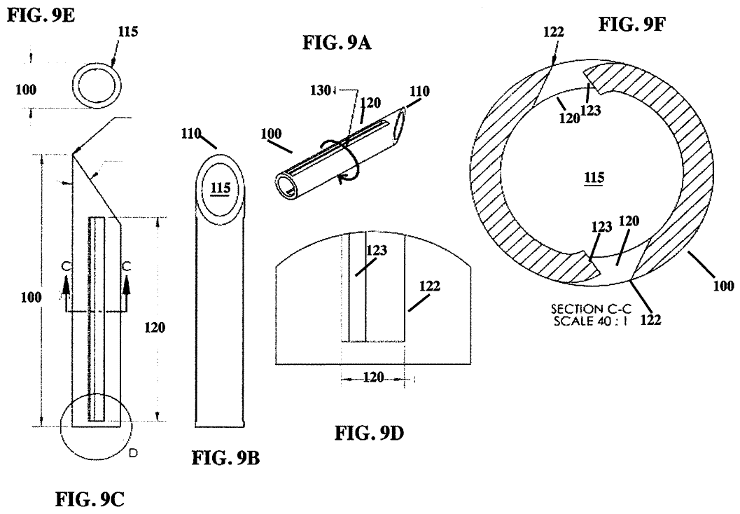

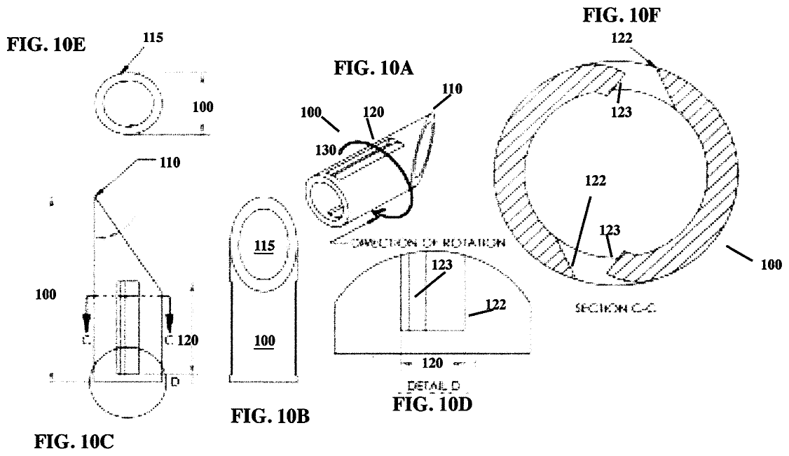

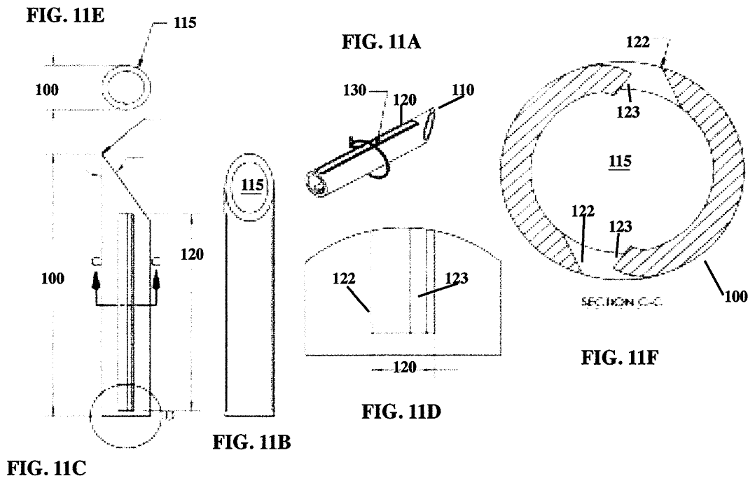

Referring now to FIGS. 9A through 9F, another alternative embodiment is shown. As shown in FIG. 9A, the probe 100 has an extended opening 120 along a significant portion of the length of the probe 100, e.g. 9 mm opening length on a 12 mm probe. The probe 100 preferably has two extended openings 120 that are approximately 180 degrees in opposition on the perimeter of the probe 100 (as shown in FIG. 9F). Preferably, each opening 120 has a sharpened leading edge 122 and a blunt opposite edge 123. The probe can be rotated so the leading edge 122 can slice through plaque in the occlusion 300. The rotational speed can be varied and controlled by the surgeon with a high torque electric motor (not shown). Plaque shaved by the probe 100 can be suctioned to a vial (not shown) at the end of the catheter 200. The direction of rotation 130 as shown in FIG. 9A is preferably clockwise. Preferably, the direction of the rotation of the probe 100 can also be reversed (counterclockwise) by the surgeon if needed to clear the openings 120. The probe 100 is preferably circumferentially laser welded at a second end to the guide wire 210 to achieve a strong attachment. This probe can be used in lengths from 5.0 mm to 12.0 mm. A shorter alternative embodiment is shown in FIGS. 10A through 10F where clockwise rotation is used to clear the openings. An alternative embodiment for counterclockwise rotation for plaque removal is in FIGS. 11A-11F. The counterclockwise rotation shown in FIG. 12A facilitates clearing of the openings 120 for the embodiment of 12A to 12F.

Preferably, the guide catheter 200 is an 8Fr (French, 1Fr=0.333 mm) JL 3.5. This catheter size is 2.67 mm in diameter and is typically suitable for movement in arteries. Other catheter sizes can be used for this application. An aspiration pump (not shown) is preferably connected to the catheter 200 and the guide wire 210 that is 0.014 inches BMW; PT2 of moderate support. The pump creates the aspiration required to suction plaque built up behind the endothelium. The needle tip 110 can preferably be re-oriented in situ by the surgeon as the level of plaque drops. Additionally, the probe 100 can be reinserted in the endothelium at another location to suction additional plaque. For the aspiration pump, Ohio Medical, among other suppliers, offers pumps that can be utilized for this application e.g. model Care-E-Vac EMS Part #5320216 800cc Canister, a portable unit with a 630 mm Hg of suction capability. Triumph Medical Equipment has a Devilbiss model with 533 mmHg of suction capability. These pumps work on 110 Volts or 220 Volts AC.

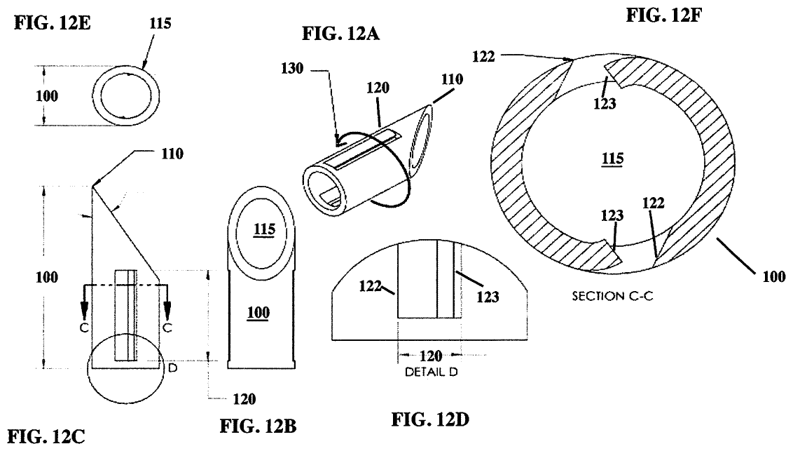

Referring now to FIG. 13, another alternative embodiment of the micro probe 100 is shown. Plaque is typically at normal body temperature, e.g. 98.4F (37C). The alternative embodiment of the micro probe shown in FIG. 13 preferably further comprises an electric heating element 140. The heating element 140 is used to heat plaque and soften tissue near the probe 100. The heating element 140 comprises electric resistance that preferably converts AC current 110 Volts (220 Volts as required) into heat. The preferred range for the heating element is 40 degrees (C°) to 70 degrees (C°). Such heating facilitates removal and suction of plaque such as from occlusion 300, particularly when plaque has hardened but not solidified.

Preferably, the temperature of the heating element 140 can be monitored by a temperature sensor (not shown) such as a modified Wiedmann TS2P, which has a range of −200 C to 300 C with an accuracy of +/−0. 2C. The sensor preferably has a sensing pad measuring 0.3 mm×0.3 mm that can stop current to the heating element 140 and prevent its temperature from increasing to excessive levels. The heating element 140 is selectively engaged by the surgeon. Preferably, the temperature of the heating element is visible to the surgeon on a screen (not shown). Additionally, and preferably, an audible signal can alert the surgeon when a pre-selected temperate of the heating element 140 has been reached. Preferably, the heating element 140 can be turned on or off by a rotational switch (not shown) that permits the increase, decrease or maintenance of a selected temperature of the heating element 140. Preferably, the heating element also has a “kill switch” (not shown) accessible by the surgeon for immediate shut-off of the heating element 140.

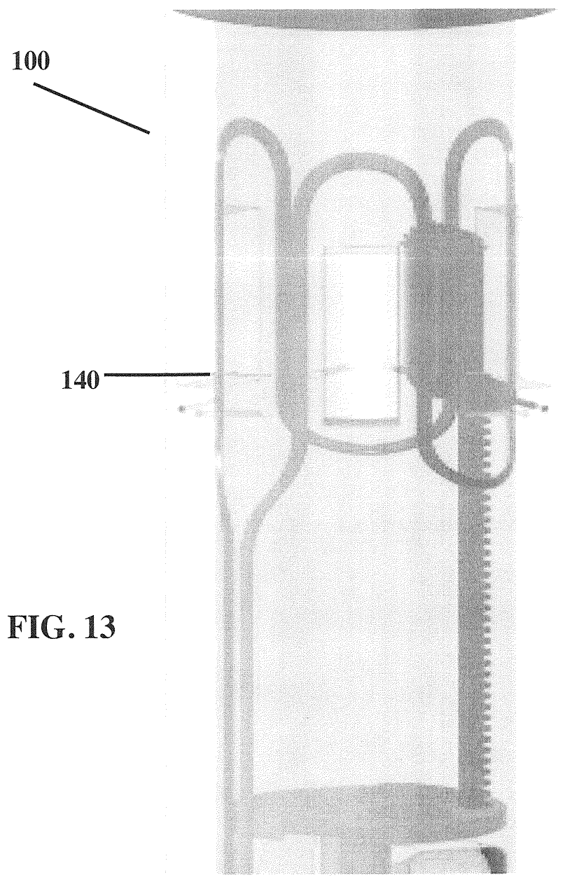

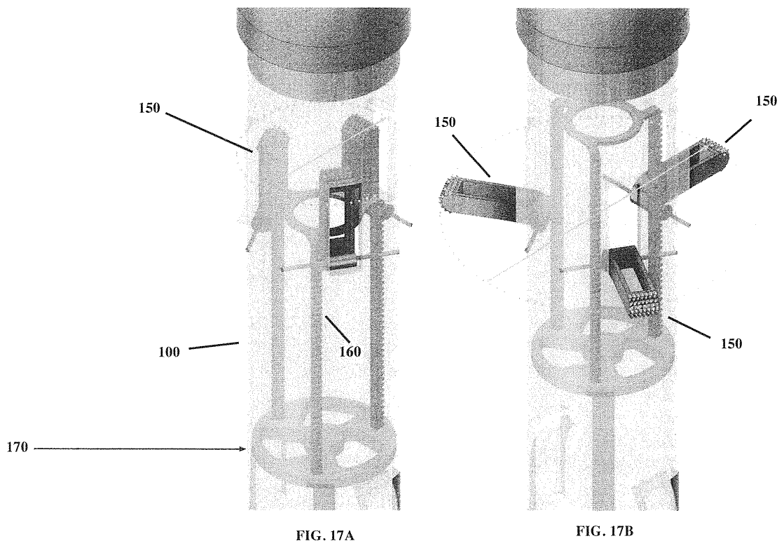

Referring now to FIG. 14, another alternative embodiment is shown. The probe 100 in FIG. 14 is preferably used when the occlusion 300 comprises hard calcium in higher quantities than typical. Accordingly, a probe 100 that permits scraping of the occlusion 300 is preferred. Preferably, as shown in FIGS. 17A and 17B, the probe 100 has three (3) retractable arms 150 that can be deployed at varying angles to facilitating the scraping of calcium build-up in an occlusion. Each arm 150 also preferably has a spiked end 155. The probe 100 shown in FIG. 14 is preferably rotatable by hand via the guide wire 210 by the surgeon. Each retractable arm 150 preferably has a rack and pinion configuration 160 to move and deploy the arms 150 at different angles as required. FIG. 14 shows the retractable arm 150 in 90-degree, 45-degree, and retracted positions. However, the arm 150 can preferably be positioned into any angle from 0 degrees (retracted) to 90 degrees to reach built-up calcium in an artery. The rack and pinion configuration 160 is preferably attached to a perforated disk 170 mounted to the guide wire 210. This allows material to be scraped and suctioned from the occlusion 300 to be pumped out of the patient while allowing rotation of the disk 170 for adjustment of the arms 150.

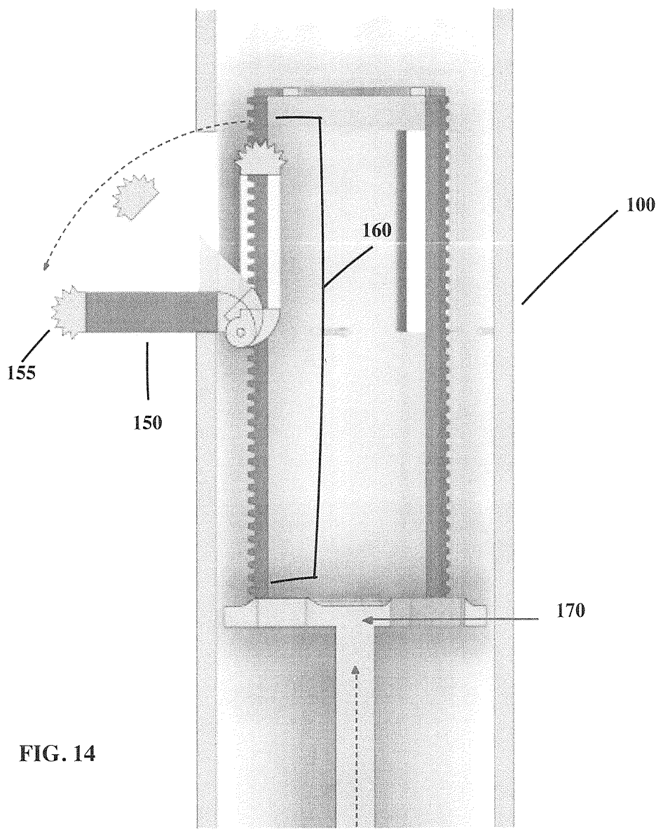

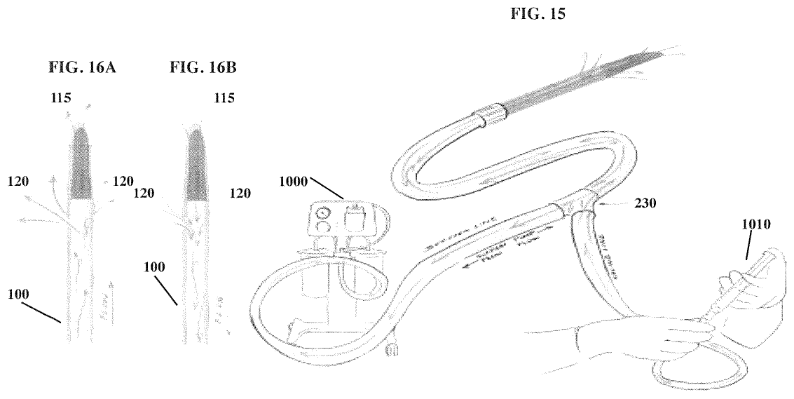

Referring now to FIGS. 15, 16A and 16B, another alternative embodiment of a micro probe system is shown. Preferably, the probe 100 has the capability to discharge a variety of substances from its orifices 120 to aid in the removal of plaque. Often, once soft plaque is removed from an occlusion, hardened plaque stuck to the walls of an artery is exposed. Similar to thrombolytic therapy where a blood clot is dissolved by injecting a drug into the affected artery, a solution 900, e.g. saline, can be pumped via an outside pump 1000 into micro probe 100 for release on to the plaque to soften the plaque and facilitate its removal.

The solution 900 is not limited to saline. For example, citric acid in a diluted solution can be sprayed from the probe 100 on to plaque. Upon reacting with the citric acid, calcium in plaque will typically break down from artery walls and be dissolved in the solution 900. That solution is then suctioned out via the probe 100, catheter 200 and pump 1000 configuration. Preferably, a catheter return line 220 has a check valve 230 to block suctioned fluid from returning to where the fresh fluid is stored, e.g. a syringe 1010. FIG. 16A shows solution 900 flowing from the probe 100 at the orifice 120 and opening 115. FIG. 16B shows solution 900 suctioned out via the orifice 120 and opening 115 into the catheter 200.

The system shown in FIGS. 15, 16A and 16B can also be utilized to deliver dry ice pellets (not shown) by using a high-pressure pump 1000 designed to handle the delivery of dry ice pellets. Dry ice is frozen carbon dioxide CO2 such as the type that has been approved by the FDA for cleaning food items such as fruits and vegetables. Pressurized by the pump 1000 to 40 psi, dry ice pellets of 1.0 mm diameter are propelled at plaque in the occlusion 300 to pulverize the plaque. Once the plaque is pulverized, it is suctioned out of the occlusion via the probe 100 to facilitate removal of the plaque via the opening 115 and orifices 120.

Thus, an improved micro probe for plaque removal is described above that can remove soft and hard plaque from a patient's arterial walls. In each of the above embodiments, the different positions and structures of the present invention are described separately in each of the embodiments. However, it is the full intention of the inventors of the present invention that the separate aspects of each embodiment described herein may be combined with the other embodiments described herein. Those skilled in the art will appreciate that adaptations and modifications of the just-described preferred embodiment can be configured without departing from the scope and spirit of the invention. Therefore, it is to be understood that, within the scope of the appended claims, the invention may be practiced other than as specifically described herein.

Various modifications and alterations of the invention will become apparent to those skilled in the art without departing from the spirit and scope of the invention, which is defined by the accompanying claims. It should be noted that steps recited in any method claims below do not necessarily need to be performed in the order that they are recited. Those of ordinary skill in the art will recognize variations in performing the steps from the order in which they are recited. In addition, the lack of mention or discussion of a feature, step, or component provides the basis for claims where the absent feature or component is excluded by way of a proviso or similar claim language.

While various embodiments of the present invention have been described above, it should be understood that they have been presented by way of example only, and not of limitation. Likewise, the various diagrams may depict an example architectural or other configuration for the invention, which is done to aid in understanding the features and functionality that may be included in the invention. The invention is not restricted to the illustrated example architectures or configurations, but the desired features may be implemented using a variety of alternative architectures and configurations. Indeed, it will be apparent to one of skill in the art how alternative functional, logical or physical partitioning and configurations may be implemented to implement the desired features of the present invention. Also, a multitude of different constituent module names other than those depicted herein may be applied to the various partitions. Additionally, with regard to flow diagrams, operational descriptions and method claims, the order in which the steps are presented herein shall not mandate that various embodiments be implemented to perform the recited functionality in the same order unless the context dictates otherwise.

Although the invention is described above in terms of various exemplary embodiments and implementations, it should be understood that the various features, aspects and functionality described in one or more of the individual embodiments are not limited in their applicability to the particular embodiment with which they are described, but instead may be applied, alone or in various combinations, to one or more of the other embodiments of the invention, whether or not such embodiments are described and whether or not such features are presented as being a part of a described embodiment. Thus, the breadth and scope of the present invention should not be limited by any of the above-described exemplary embodiments.

Terms and phrases used in this document, and variations thereof, unless otherwise expressly stated, should be construed as open ended as opposed to limiting. As examples of the foregoing: the term “including” should be read as meaning “including, without limitation” or the like; the term “example” is used to provide exemplary instances of the item in discussion, not an exhaustive or limiting list thereof; the terms “a” or “an” should be read as meaning “at least one,” “one or more” or the like; and adjectives such as “conventional,” “traditional,” “normal,” “standard,” “known” and terms of similar meaning should not be construed as limiting the item described to a given time period or to an item available as of a given time, but instead should be read to encompass conventional, traditional, normal, or standard technologies that may be available or known now or at any time in the future. Likewise, where this document refers to technologies that would be apparent or known to one of ordinary skill in the art, such technologies encompass those apparent or known to the skilled artisan now or at any time in the future.

A group of items linked with the conjunction “and” should not be read as requiring that each and every one of those items be present in the grouping, but rather should be read as “and/or” unless expressly stated otherwise. Similarly, a group of items linked with the conjunction “or” should not be read as requiring mutual exclusivity among that group, but rather should also be read as “and/or” unless expressly stated otherwise. Furthermore, although items, elements or components of the invention may be described or claimed in the singular, the plural is contemplated to be within the scope thereof unless limitation to the singular is explicitly stated.

The presence of broadening words and phrases such as “one or more,” “at least,” “but not limited to” or other like phrases in some instances shall not be read to mean that the narrower case is intended or required in instances where such broadening phrases may be absent. The use of the term “module” does not imply that the components or functionality described or claimed as part of the module are all configured in a common package. Indeed, any or all of the various components of a module, whether control logic or other components, may be combined in a single package or separately maintained and may further be distributed across multiple locations.

As will become apparent to one of ordinary skill in the art after reading this document, the illustrated embodiments and their various alternatives may be implemented without confinement to the illustrated examples. For example, block diagrams and their accompanying description should not be construed as mandating a particular architecture or configuration.

The previous description of the disclosed embodiments is provided to enable any person skilled in the art to make or use the present invention. Various modifications to these embodiments will be readily apparent to those skilled in the art, and the generic principles defined herein may be applied to other embodiments without departing from the spirit or scope of the invention. Thus, the present invention is not intended to be limited to the embodiments shown herein but is to be accorded the widest scope consistent with the principles and novel features disclosed herein.

What is claimed is:

1. A micro probe for plaque removal comprising:
a length, a first end and a second end;
where the first end comprises a needle tip and the second end is attachable to a guide wire and the probe is insertable and retractable in a catheter.

2. The micro probe of claim 1 where the micro probe further comprises a perimeter around a diameter, an opening and a plurality of perimeter openings.

3. The micro probe of claim 2 where the plurality of perimeter openings comprises a first row of perimeter openings closer to the second end and a second row of perimeter openings closer to the first end.

4. The micro probe of claim 1 where each perimeter opening of the plurality of perimeter openings has a sharp leading edge and a blunt opposite edge and the micro probe can be rotated when attached to the guide wire.

5. A micro probe for plaque removal attached to a guide wire in a catheter where the catheter is attached to a pump for suction and aspiration, the micro probe comprising:
a length, a perimeter around a diameter, a first end and a second end; where the first end comprises a needle tip and an opening and the second end is attachable to the guide wire and the probe is insertable and retractable into the catheter;
a plurality of perimeter openings, where each perimeter opening of the plurality of perimeter openings has a sharp edge and a blunt opposite edge and the micro probe can be rotated by the guide wire.

6. The micro probe of claim 5 where the opening can contain a dry ice pellet that can be impelled by the pump.

7. The micro probe of claim 5 where the catheter is attached to a solution reservoir with a solution and the pump can aspirate the solution through the opening and plurality of openings of the micro probe.

8. The micro probe of claim 5 further comprising a heating element and a temperature sensor, where the heating element can adjustably increase the temperature of the micro probe to a range of 40 to 70 degrees Celsius.

9. A micro probe for plaque removal attached to a guide wire in a catheter where the catheter is attached to a pump for suction and aspiration, the micro probe comprising:
a length, a perimeter around a diameter, a first end and a second end; where the first end comprises a needle tip and an opening and the second end is attachable to the guide wire and the probe is insertable and retractable into the catheter;
a plurality of perimeter openings, where each perimeter opening of the plurality of perimeter openings has a scraping arm that can be extended from and retracted to the perimeter opening by a rack and pinion structure attached to a perforated disk;
where the perforated disk is rotatable by the guide wire to extend and retracted each scraping arm.

10. The micro probe of claim 9 where each scraping arm has a spiked end.

11. The micro probe of claim 9 where each scraping arm can be extended to 90 degrees from the perimeter opening.

12. The micro probe of claim 9 where the micro probe comprises at least three scraping arms.
